(19) United States
(12) Patent Application Publication
(10) Pub. No.: US 2014/0104311 A1
(43) Pub. Date: Apr. 17, 2014

(54) MEDICAL IMAGE DISPLAY METHOD USING VIRTUAL PATIENT MODEL AND APPARATUS THEREOF

(71) Applicant: Infinitt Healthcare Co., Ltd., Seoul (KR)

(72) Inventors: Seung Chul Park, Seoul (KR); Min Sung Kim, Seoul (KR)

(73) Assignee: Infinitt Healthcare Co., Ltd., Seoul (KR)

(21) Appl. No.: 14/052,163

(22) Filed: Oct. 11, 2013

(30) Foreign Application Priority Data
Oct. 12, 2012 (KR) 10-2012-0113255

(51) Int. Cl. A61B 5/00

(52) U.S. Cl. CPC A61B 5/7425; A61B 5/743; A61B 5/7435
USPC 345/629

(57) ABSTRACT

Disclosed herein are a method and apparatus for displaying medical images using a virtual patient model. The apparatus includes a processor, the processor includes an image acquisition unit, a captured body part determination unit, a determination unit, and a display (control) unit. The image acquisition unit acquires one or more medical images of a patient. The captured body part determination unit determines the captured body part of each of the acquired one or more medical images. The determination unit determines a human body model diagram corresponding to unique information of the patient among a plurality of predetermined human body model diagrams. The display unit places and displays the one or more medical images on the determined captured body parts of the determined human body model diagram.

### CROSS-REFERENCE TO RELATED APPLICATION

This application claims under 35 U.S.C. §119(a) the benefit of Korean Application No. 10-2012-0113255 filed Oct. 12, 2012, which is incorporated herein by reference.

### TECHNICAL FIELD

The present invention relates, in general, to the display of medical images and, more particularly, to a method and apparatus for displaying medical images using a virtual patient model, which is capable of varying a display orientation based on information about medical images of a patient using a virtual human body model diagram suitable for the patient, thereby enabling a user (a doctor) to intuitively check various and important images of the patient.

The present invention was contrived based on research that was conducted as part of the knowledge economy innovation project (fundamental industrial technology development project) of the Korean Ministry of Knowledge Economy and the Korea Evaluation Institute of Industrial Technology (project management number: 10038419, project title: Intelligent

### BACKGROUND ART

Today radiation or magnetic apparatuses, such as a computerized tomography (CT) scanner, a magnetic resonance imaging (MRI) scanner and an X-ray machine, are being widely used as diagnostic equipment. Traditionally, radiologists use equipment, such as an X-ray machine that generates images on films.

In general, various medical images of a patient are acquired using diagnostic equipment, and the medical status of the patient is analyzed and diagnosed using the medical images.

An X-ray machine acquires many X-ray images of a region of interest from different angles, and a doctor diagnoses the medical status of a patient using the acquired various images.

In the medical field, it is common to store, distribute and check images in digital form using computer technology. Picture Archival and Communication Systems (PACSs) are being widely used. In general PACS applications, image data is acquired using medical image apparatuses, such as CT and MRI scanners, and the acquired image data is stored in the form of computer files. The size of an image data file varies depending on the size and resolution of an image. General diagnostic chest X-ray image files have a size of 10 MB.

Such image data files are generated in a basic format or a widely used format. The image format that is widely used in the medical field is a Digital Imaging and Communications in Medicine (DICOM) format. DICOM image data files may be distributed to viewing stations capable of converting DICOM image files into high-resolution images over a network.

In conventional technology, as illustrated in an example of FIG. 1, information stored for each patient is provided by matching the ID of the patient with information about medical images of the patient on a text basis. In order to check detailed information about the patient, a corresponding patient item 110 should be selected, and then detailed information 120 about the selected item 110 should be checked in a separate display region in the form of text. That is, the conventional technology is problematic in that a user cannot intuitively check medical status and medical images of a patient because information is provided on a text basis.

Accordingly, there is a demand for a method of providing information about a patient and captured medical images to a user (a doctor) in an intuitive manner.

### SUMMARY OF THE DISCLOSURE

Accordingly, the present invention has been made keeping in mind the above problems occurring in the prior art, and an object of the present invention is to provide a method and apparatus for displaying medical images using a virtual patient model, which enable medical images of a patient to be placed on a virtual human body model diagram suitable for the patient in accordance with captured body parts thereof, thereby enabling a user (a doctor) to intuitively check various and important images of the patient.

More specifically, the present invention is configured to control the display orientation of a human body model diagram of a patient based on user input or the location of a lesion included in medical images of a patient, thereby enabling a user to clearly view important images.

Another object of the present invention is to provide a method and apparatus for displaying medical images using a virtual patient model, which can provide information about a patient and medical images of the patient placed on captured body parts of a human body model diagram through a user interface, thereby enabling the medical status of the patient to be easily determined using the medical images of the patient and also enabling a user to conveniently check the medical information of the patient.

In accordance with an aspect of the present invention, there is provided a method of displaying medical images using a virtual patient model, including acquiring one or more medical images of a patient; determining the captured body part of each of the acquired one or more medical images; determining whether a lesion exists on the one or more medical images; if the lesion is present, controlling a display orientation of a predetermined human body model diagram of the patient while taking into account a location of the lesion; and disposing and displaying the one or more medical images on the determined captured body parts of the human body model diagram whose display orientation has been controlled.

The displaying may include reconstructing the one or more medical images in accordance with the display orientation of the human body model diagram; and disposing and displaying the reconstructed one or more medical images on the determined captured body parts.

The displaying may include adjusting the size of each of the one or more medical images to a predetermined scale; and disposing and displaying the one or more medical images on the determined captured body parts.

The displaying may include, if a plurality of medical images corresponds to a same captured body part, disposing and displaying one of the plurality of medical images on a corresponding captured body part while taking into account at least one of types of images and imaging times (or capture times) of the images and predetermined priorities.

The displaying may include displaying the number of the plurality of medical images.

The displaying may include displaying thumbnail images corresponding to the plurality of medical images in a separate display region.

The displaying may include, if the size of any of the captured body parts is smaller than a reference size (or a specific/critical size), displaying a predetermined diagram or color in the corresponding captured body part.

The controlling of the display orientation may include, if a plurality of lesions exists, determining one lesion among the plurality of lesions while taking into account at least one of predetermined priorities predetermined based on types of lesions and a field of profession of a user, and controlling the display orientation of the human body model diagram while taking into account a location of the determined lesion.

The method may further include determining a human body model diagram corresponding to the unique information of the patient among a plurality of predetermined human body model diagrams, and the controlling may include controlling the display orientation of the determined human body model diagram corresponding to the unique information of the patient.

In accordance with another aspect of the present invention, there is provided a method of displaying medical images using a virtual patient model, including acquiring one or more medical images of a patient; determining a captured body part of each of the acquired one or more medical images; determining a human body model diagram corresponding to unique information of the patient among a plurality of predetermined human body model diagrams; and disposing and displaying the one or more medical images on the determined captured body parts of the determined human body model diagram.

The determined human body model diagram can be rotated by a user (user input or user command), and the displaying may include, if the display orientation of the human body model diagram is changed by the user, reconstructing the one or more medical images in accordance with the changed display orientation, and disposing and displaying the reconstructed one or more medical images on the determined captured body parts.

The method may further include determining whether a lesion exists on the one or more medical images, and, if a lesion is present (or exists), controlling a display orientation of the determined human body model diagram while taking into account a location of the lesion, and the displaying may include disposing and displaying the one or more medical images on the determined captured body parts of the human body model diagram whose display orientation has been controlled.

In accordance with still another aspect of the present invention, there is provided an apparatus for displaying medical images using a virtual patient model, including an image acquisition unit configured to acquire one or more medical images of a patient; a captured body part determination unit configured to determine a captured body part of each of the acquired one or more medical images; a determination unit configured to determine a human body model diagram corresponding to unique information of the patient among a plurality of predetermined human body model diagrams; and a display unit configured to place and display the one or more medical images on the determined captured body parts of the determined human body model diagram.

The apparatus may further include a checking unit configured to check whether a lesion is present (or exists) on the one or more medical images, and a control unit configured to, if the lesion is present, control a display orientation of the determined human body model diagram while taking into account a location of the lesion, and the display unit may place and display the one or more medical images on the determined captured body parts of the human body model diagram whose display orientation has been controlled.

### BRIEF DESCRIPTION OF THE DRAWINGS

The above and other objects, features and advantages of the present invention will be more clearly understood from the following detailed description taken in conjunction with the accompanying drawings, in which:

FIG. 1 is a diagram illustrating a conventional example of providing information about medical images on a text basis;

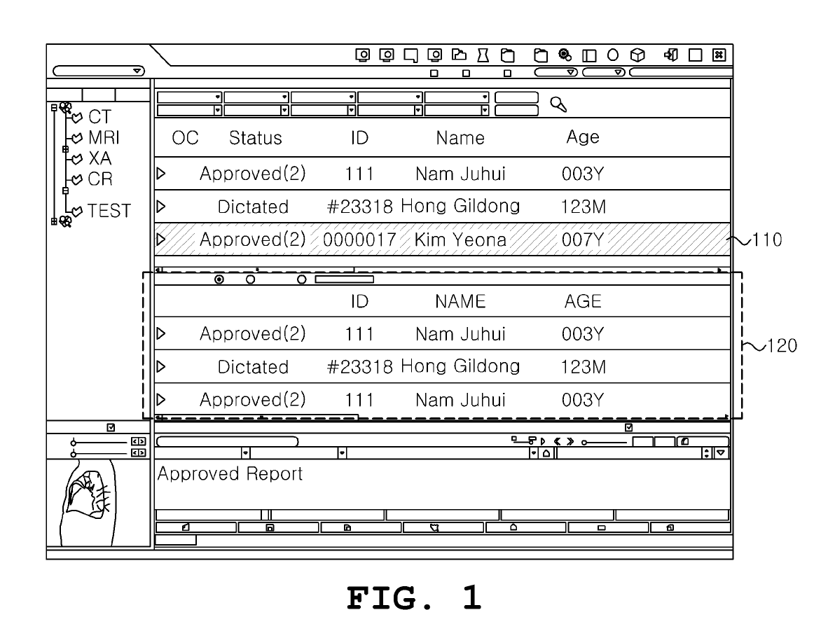

FIG. 2 is an operation flowchart of a method of displaying medical images using a virtual patient model in accordance with an embodiment of the present invention;

FIG. 3 is an operation flowchart illustrating step S290 of FIG. 2 in accordance with an embodiment of the present invention;

FIG. 4 is an operation flowchart illustrating a process that may be performed after step S340 of FIG. 3 in accordance with an additional embodiment of the present invention;

FIG. 5 is an operation flowchart illustrating step S290 of FIG. 2 in accordance with another embodiment of the present invention;

FIG. 6 is a diagram illustrating an example in which a human body model diagram and medical images of a patient whose display orientation has been controlled;

FIG. 7 is a diagram illustrating an example in which medical images are placed and displayed on a human body model diagram of a patient;

FIG. 8 is a diagram illustrating an example in which thumbnail images corresponding to a plurality of medical images are displayed if the plurality of medical images corresponds to the same captured body part;

FIG. 9 is an operation flowchart illustrating a method of displaying medical images using a virtual patient model in accordance with another embodiment of the present invention;

FIG. 10 is a diagram illustrating an example of a user interface that provides information about a patient and medical images of the patient in an intuitive manner; and

FIG. 11 is a diagram illustrating the configuration of an apparatus for displaying medical images using a virtual patient model in accordance with an embodiment of the present invention.

### DETAILED DESCRIPTION OF THE DISCLOSURE

Embodiments of the present invention will be described in detail below with reference to the accompanying drawings. Reference now should be made to the elements of drawings, in which the same reference numerals are used throughout the different drawings to designate the same elements. In the following description, detailed descriptions of known elements or functions that may unnecessarily make the gist of the present invention obscure will be omitted.

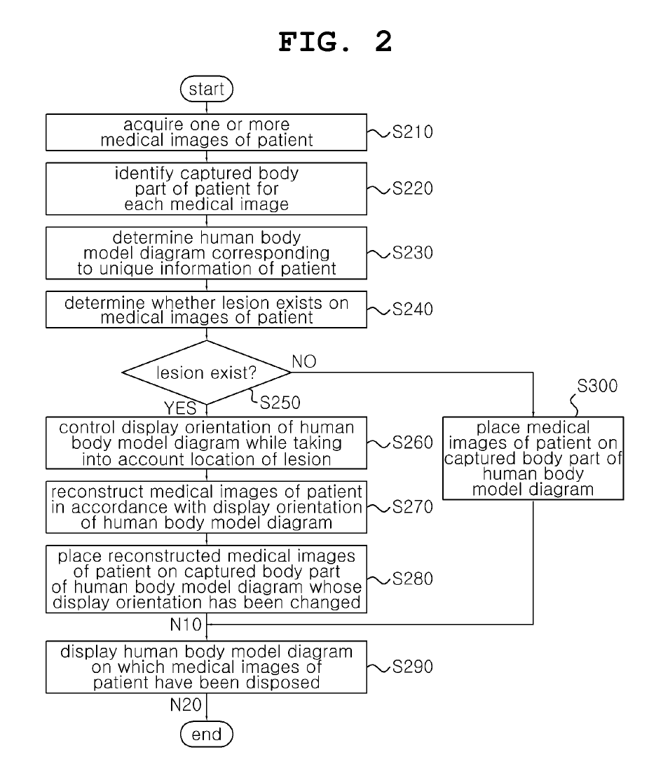

A method and apparatus for displaying medical images using a virtual patient model in accordance with embodiments of the present invention will be described below in detail below with reference to FIGS. 2 to 11.

FIG. 2 is an operation flowchart of a method of displaying medical images using a virtual patient model in accordance with an embodiment of the present invention.

Referring to FIG. 2, in the method in accordance with this embodiment of the present invention, first, one or more medical images of a patient are acquired at step S210, and the body part of the patient corresponding to each of the acquired medical images of the patient is determined at step S220.

In this case, the medical images of the patient may be acquired using a PACS or the like, and the body part of the patient corresponding to each of the acquired medical images of the patient may be determined using medical image data stored for each of the medical images, for example, information stored in a DICOM header. A DICOM header includes information about target body part of medical image, for example, head, chest, stomach, arms, legs and whole body, etc. It will be apparent that the body part of the patient corresponding to each of the acquired medical images of the patient may be determined by performing image processing and analysis on each of the medical images of the patient. It means, an image processing system may identify and determine the target body part of a medical image by performing segmentation, reconstruction, etc.

The medical images of the patient may be 3D medical images (multi-planar reconstruction (MPR), maximum intensity projection (MIP) or volume rendering (VR) images, or the like) that are taken by a medical imaging apparatus, such as a CT or MRI scanner or the like, and are then reconstructed, or 2D medical images that are taken by a medical imaging apparatus, such as an X-ray machine. It will be apparent that the medical images of the patient are not limited to the above-described medical images but may include all types of medical images of the patient that are taken by a positron emission tomography (PET) scanner, an angiographic machine, a computed radiography (CR)/digital radiography (DR) scanner, fundoscopy equipment, an electrocardiogram (EKG) scanner, etc.

Then a virtual human body model diagram of the patient is determined. One of a plurality of previously created human body model diagrams corresponding to the unique information of the patient is determined to be the human body model diagram of the patient at step S230.

In this case, the plurality of human body model diagrams may be created based on age, gender, height, body weight and/or the like. The human body model diagram of the patient may be selected from among the plurality of human body model diagrams based on age, gender, height, body weight and/or the like.

In this case, the plurality of human body model diagrams may be created using statistical data about a plurality of patients.

At step S240, it is checked whether a lesion is included in the medical images of the patient acquired at step S210, and at step S250, it is determined whether a lesion exists in the medical images of the patient through a determination process.

If, as a result of the determination at step S250, it is determined that a lesion is not present in the medical images of the patient, that is, there is no medical image including a lesion, the medical images of the patient are placed on the human body model diagram of the patient in a basic display orientation, for example, a frontward orientation. That is, at step S300, the medical images are placed on the corresponding captured body parts determined at step S220, and at step S290, the human body model diagram on which the medical images of the patient have been placed on the captured body parts is displayed.

In this case, if any of the medical images does not accurately match a corresponding captured body part, a user may directly match them with each other through user input.

In contrast, if, as a result of determination at step S250, it is determined that there is a medical image including a lesion among the medical images of the patient, the display orientation of the human body model diagram of the patient is controlled depending on the location of the lesion at step S260.

In this case, the controlled display orientation of the human body model diagram may be an orientation in which the lesion can be easily seen on the medical image including the lesion. The orientation in which the lesion can be easily seen on the medical image may be stored for each captured body part, each lesion location, each gender, or each age in advance. For example, if a first lesion can be easily seen when the corresponding medical image is rotated in a leftward orientation over 30 degrees, the display orientation of the first lesion is an orientation that is rotated over 30 degrees to the left.

Meanwhile, a plurality of lesions may be present on the medical images of the patient. If a plurality of lesions is present, one lesion may be determined among the plurality of lesions based on at least one of priorities predetermined based on the types of lesions and the field of profession of a user (doctor). It will be apparent that if a plurality of lesions is present, the notification of the presence of the lesions may be transferred to a user, and thus the user may directly select a lesion or a medical image including a lesion.

Once the display orientation of the human body model diagram of the patient has been determined, the medical images of the patient are reconstructed such that they can be displayed in the determined display orientation at step S270.

In this case, the reconstructed medical images of the patient may be 3D medical images (MPR, MIP or VR images, or the like). In the case of 2D medical images, it is preferable to maintain the images without change regardless of the display orientation.

Once the medical images of the patient have been reconstructed in accordance with the display orientation, the reconstructed medical images of the patient are placed on the human body model diagram of the patient whose display orientation has been controlled. That is, the medical images of the patient are placed on the determined captured body parts of the human body model diagram at step S280, and the virtual human body model diagram of the patient on which the medical images of the patient have been placed is displayed at step S290.

In this case, the medical images of the patient to be placed on the human body model diagram may be adjusted to a predetermined scale, the scaled medical images may be placed on the corresponding captured body parts, and then the human body model diagram may be displayed. If any of the medical images does not accurately match a corresponding captured body part, a user may directly match them with each other.

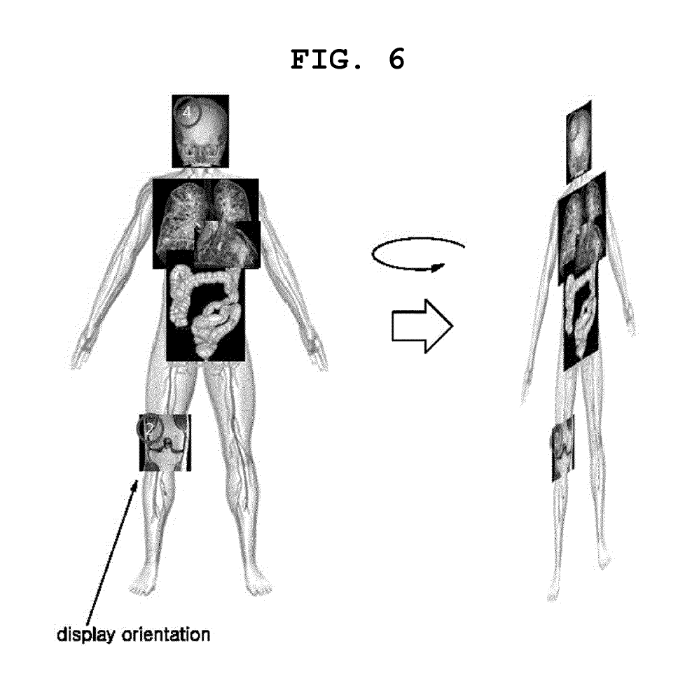

The operation of the present invention will now be described with reference to FIG. 6. As illustrated in FIG. 6, if there is no medical image including a lesion among the medical images of the patient, medical images of a patient may be placed and displayed on a human body model diagram in a basic display orientation, as in the left human body model diagram. If a lesion exists on the medical images of the patient and the orientation in which the lesion can be easily seen is not a frontward orientation but a leftward orientation, a human body model diagram whose display orientation has been rotated in a leftward orientation, and the medical images of the patient which have been reconstructed in accordance with the leftward orientation are placed on the human body model diagram, as in the right human body model diagram, and thus a lesion can be easily seen by a user (doctor).

Although the medical images of the patient have been illustrated as being rotated in terms of two dimensions in the right human body model diagram of FIG. 6, only the human body model diagram may be rotated and the medical images may be reconstructed in accordance with display orientation and placed in corresponding regions.

Furthermore, although in accordance with this embodiment of the present invention, the display orientation is controlled in accordance with the location of the lesion and the medical images are reconstructed and placed accordingly, this display orientation may not be the display orientation desired by a user, and thus the user may directly control the display orientation through user input. It will be apparent that even when the user directly controls the display orientation, the medical images of the patient should be reconstructed and placed on the controlled human body model diagram.

In this embodiment of the present invention, the human body model diagram of the patient is controlled, for example, rotated, in accordance with the display orientation and then displayed. In this case, with regard to the displayed human body model diagram, a new human body model diagram of the patient may be generated in accordance with the display orientation, or a previously generated human body model diagram may be displayed in accordance with the display orientation. Human body model diagrams with respect to regular angular intervals may be previously generated, and a human body model diagram may be generated using the previously generated human body model diagrams if the display orientation is determined to be between specific angles. If the display orientation in which a lesion can be easily checked can be determined based on the body part and the type of lesion type, human body model diagrams in display orientations based on body parts and the types of lesions may be previously generated and provided. In this case, the display orientations based on the body parts and the types of lesions may be determined based on statistical data about a number of patients.

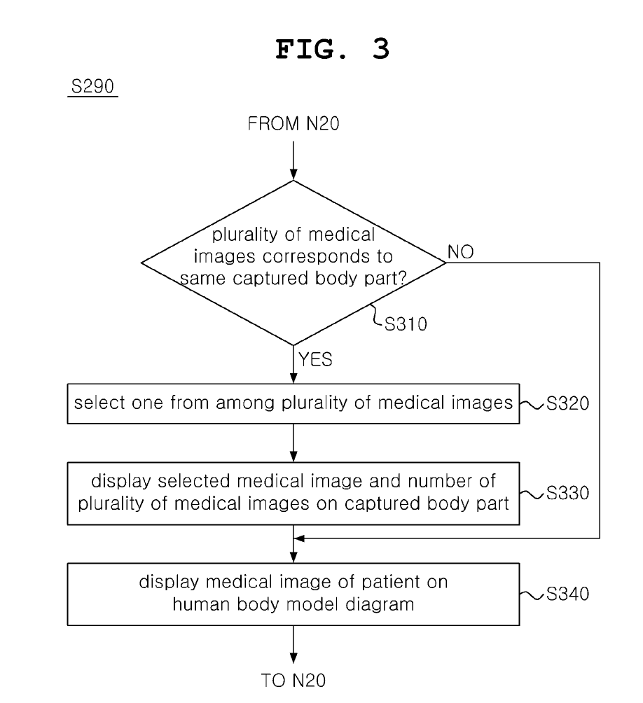

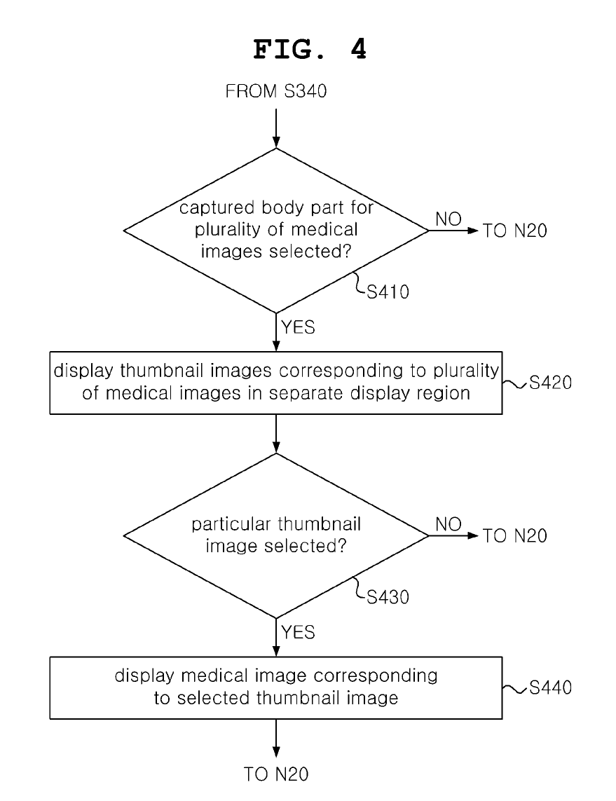

A patient may require that the same body part (or body region) be captured a plurality of times or the same body part be captured using a plurality of medical imaging apparatuses. In this case, a scheme for determining a single medical image to be placed and displayed on a human body model diagram of the patient and providing the remaining medical images should be provided. An embodiment related to this will be described with reference to FIGS. 3 and 4.

FIG. 3 is an operation flowchart illustrating step S290 of FIG. 2 in accordance with an embodiment of the present invention.

Referring to FIG. 3, step S290 of displaying a human body model diagram on which the medical images of a patient have been placed includes step S310 of determining whether a plurality of medical images corresponding to the same one of the captured body parts determined at step S220 are present among the medical images of the patients.

If, as a result of the determination at step S310, a plurality of medical images corresponding to the same body part of the patient is present, one medical image is selected from among the plurality of medical images of the corresponding captured body part at step S320, and the selected medical image is placed on the corresponding captured body part and the number of the plurality of medical images is also displayed on the corresponding captured body part at step S330.

In this case, the one medical image may be selected from among the plurality of medical images based on at least one of the types and captured times of the images and predetermined priorities. The priorities may be determined by a user (doctor) who views the medical images of the patient, or may be automatically determined based on user information, for example, the field of profession or interest of a user.

The human body model diagram of the patient, together with the selected medical image and the number of medical images corresponding to the body part or captured body part, is displayed at step S340.

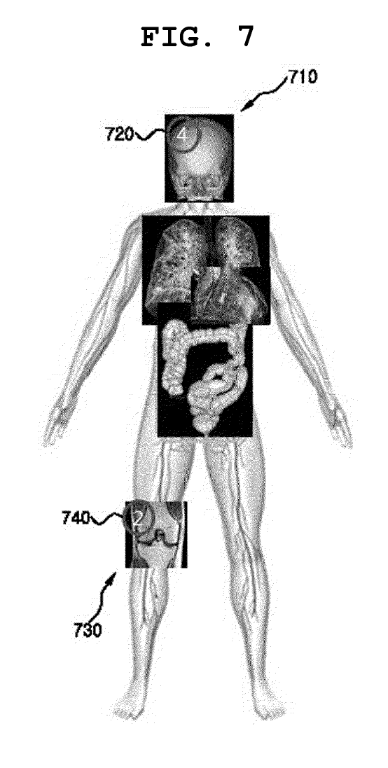

For example, when the number of medical images of the head of a patient is 4 and the number of medical images of the right knee is 2, as illustrated in FIG. 7, one medical image 710 of the four medical images of the head is displayed on the human body model diagram of the patient and the number of images 720 is also displayed. In the same manner, one medical image 730 of the two medical images of the right knee is displayed on the human body model diagram and the number of images 740 is also displayed. In this case, the number of medical images that is displayed may be the number of medical images, inclusive of a medical image that is displayed on a corresponding captured body part, or the number of medical images, exclusive of a medical image that is displayed on a corresponding captured body part.

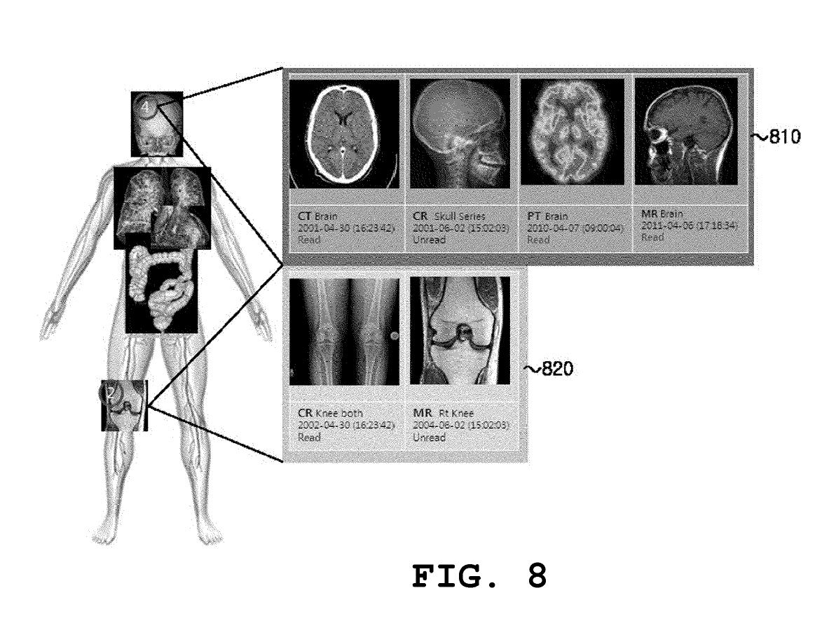

Since only one of a plurality of medical images corresponding to the same captured body part is displayed in this embodiment of the present invention, the remaining medical images may be provided in the form of thumbnail images. This will be described with reference to FIGS. 4 and 8.

FIG. 4 is an operation flowchart illustrating a process that may be performed after step S340 of FIG. 3 in accordance with an additional embodiment of the present invention.

Referring to FIG. 4, if a user selects a captured body part on which a plurality of medical images is present from the human body model diagram of the patient at step S410, the plurality of medical images present on the selected captured body part is displayed in a separate display region in the form of thumbnail images, as illustrated in the example of FIG. 8, at step S420.

That is, as illustrated in the example of FIG. 8, if the header is selected from the human body model diagram, thumbnail images corresponding to the medical images of the header are displayed in a first display region 810. Meanwhile, if the right knee is selected therefrom, thumbnail images corresponding to the medical images of the right knee are displayed in a second display region 820.

In this case, the thumbnail images may be displayed in order of captured time, capturing device type or the like. If the number of thumbnail images is larger than that of medical images that can be displayed in the display region, only thumbnail images that can be displayed in the display region are displayed and also an indication that the remaining thumbnail images are present may be visually provided.

Furthermore, a user interface or a button that allows thumbnail images of a plurality of captured body parts including a plurality of medical images to be displayed at one time may be separately provided. A user interface or a button that allows thumbnail images of the medical images of the patient to be displayed at one time may be separately provided.

If the plurality of thumbnail images is displayed at step 420 and one thumbnail image is selected from among the displayed plurality of thumbnail images at step S430, a medical image corresponding to the thumbnail image selected by the user is displayed at step S440.

In this case, the medical image corresponding to the thumbnail image may be displayed on a corresponding region of a human body model diagram, or may be displayed on a separate display region.

Furthermore, in this embodiment of the present invention, a thumbnail image may be directly selected, dragged and then placed on a specific region of the human body model diagram by the user, in which case an image that is displayed on the specific region may be the thumbnail image or a medical image corresponding to the thumbnail image.

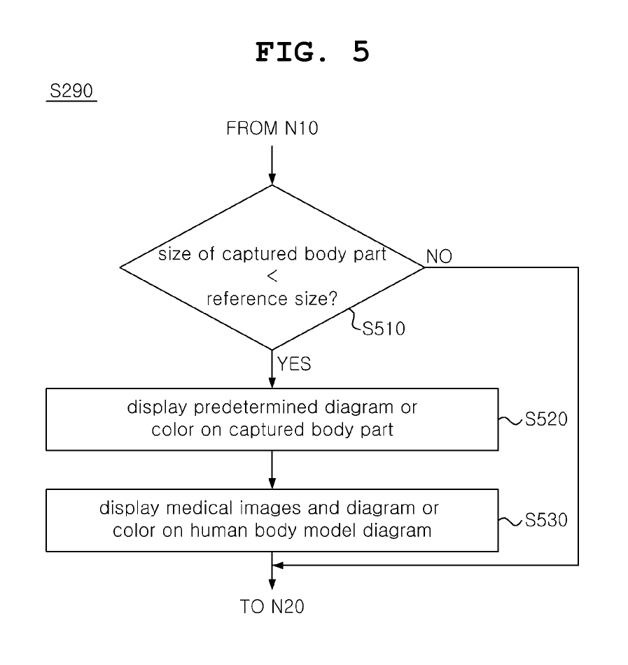

FIG. 5 is an operation flowchart illustrating step S290 of FIG. 2 in accordance with another embodiment of the present invention. This embodiment is directed to a scheme that can indicate that a medical image is present when the medical image of a captured body part cannot be directly displayed on a human body model diagram because the captured image is excessively small.

Referring to FIG. 5, step S290 of displaying a human body model diagram on which medical images have been placed includes step S510 of determining whether the size of each of the determined captured body parts of captured medical images is smaller than that of a predetermined reference size.

In this case, the reference size may vary depending on the situation.

If, as a result of the determination at step S510, it is determined that the size of one of the captured body parts is smaller than the reference size, a predetermined diagram or color is displayed on the corresponding captured body part at step S520, and the human body model diagram of the patient on which the remaining medical images of the patient and the diagram or color have been displayed is displayed at step S530.

As described above, in the method of displaying medical images using a virtual patient model in accordance with the present invention, the display orientation of a human body model diagram is determined in accordance with the location of the lesion, the human body model diagram is rotated in accordance with the determined display orientation, and medical images of a patient are reconstructed in accordance with the display orientation and placed on corresponding captured body parts of the human body model diagram. Accordingly, the overall scanned status of the patient, the actual anatomical structure of the patient and the states of one or more lesions can be checked at a glance.

Furthermore, if a plurality of medical images corresponding to a captured body part is present, thumbnail images corresponding to the plurality of medical images are displayed, the type of examination and an important image for the corresponding captured body part may be displayed. As described above, the present invention is configured to provide an intuitive user interface to a user, thereby easily providing convenience, information about a patient, and medical images of the patient to a user.

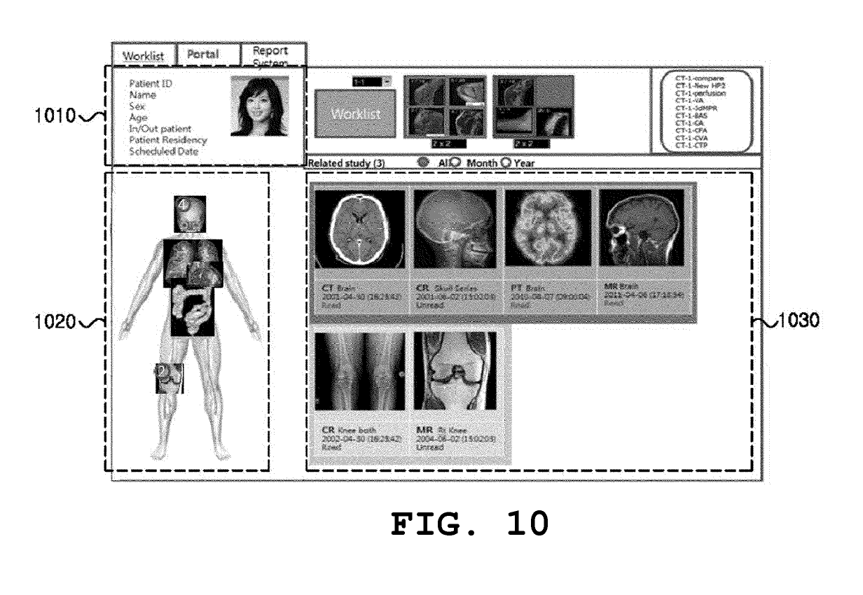

Through the above-described processes, as illustrated in FIG. 10, information about a patient 1010, a human body model diagram 1020 of the patient on which medical images have been placed, and thumbnail images 1030 may be displayed on a user viewer. It will be apparent that whether to display the thumbnail images may be determined by the user's selection.

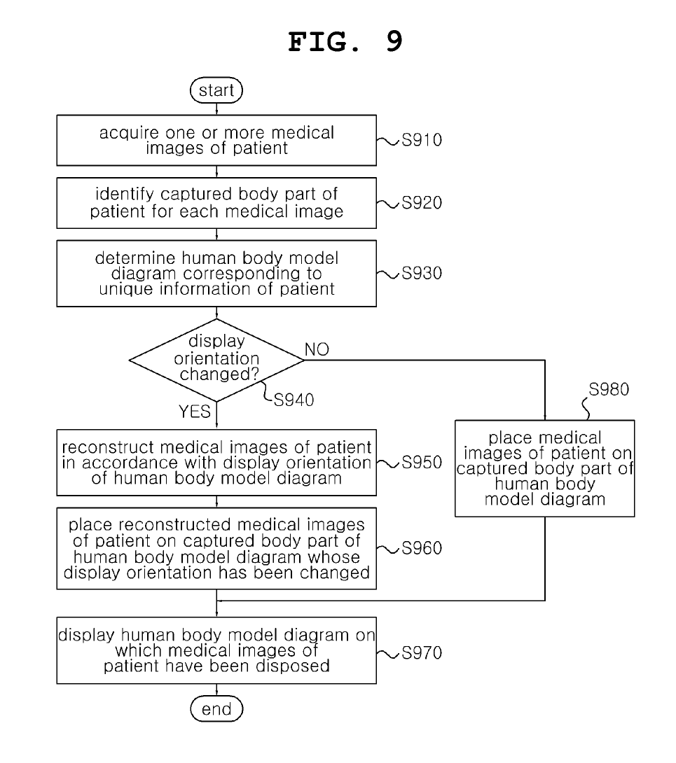

FIG. 9 is an operation flowchart illustrating a method of displaying medical images using a virtual patient model in accordance with another embodiment of the present invention.

Referring to FIG. 9, the method of the present invention includes step S910 of acquiring one or more medical images of a patient, and step S920 of determining the body part of the patient corresponding to each of the acquired medical images of the patient.

In this case, the medical images of the patient may be acquired using a PACS or the like. The body part of the patient corresponding to each of the acquired medical images of the patient may be determined using medical image data stored for each of the medical images, for example, information stored in a DICOM header, or may be determined by performing image processing and analysis on each of the medical images of the patient.

In this case, the medical images of the patient may include all types of medical images of the patient that are taken by a CT scanner, an MRI scanner, a PET scanner, an angiographic machine, a CR/DR scanner, fundoscopy equipment, an EKG scanner, and the like.

Then a virtual human body model diagram of the patient is then determined. One of a plurality of previously created human body model diagrams corresponding to the unique information of the patient is determined to be the human body model diagram of the patient at step S930.

In this case, the unique information of the patient may include information about the age, gender, height and body weight, and all other types of data that can be used to identify the patient.

Thereafter, whether to change the display orientation of the human body model diagram is determined at step S940. That is, whether to display a basic human body model diagram, for example, a frontward human body model diagram, or a human body model diagram viewed from a different angle, is determined.

If, as a result of the determination at step S940, the display orientation is not determined to be changed, the medical images of the patient are placed on a human body model diagram in a basic display orientation. At step S980, the medical images are placed on the captured body parts determined at step S920, and at step S970, the human body model diagram on which the medical images of the patient have been placed on the captured body parts is displayed.

In this case, the display orientation may be determined through user input, or may be changed by determining whether a lesion is included in the medical images of the patient and taking into account the location of the lesion if the lesion is included in the medical images.

For example, the display orientation may be changed in such a way that a user rotates the human body model diagram of the patient, or may be changed by determining whether a lesion is included in the medical images of the patient and taking into account the location of the lesion if the lesion is included in the medical images. Since controlling or changing the display orientation of the human body model diagram while taking into account the location of the lesion has been previously described, a detailed description thereof is omitted.

Once the display orientation of the human body model diagram of the patient has been determined, the medical images of the patient are reconstructed such that they can be displayed in the determined display orientation at step S950.

In this case, the reconstructed medical images of the patient may be 3D medical images (MPR, MIP or VR images, or the like). In the case of 2D medical images, it is preferable to maintain the images without change regardless of the display orientation.

Once the medical images of the patient have been reconstructed in accordance with the display orientation, the reconstructed medical images of the patient are placed on the human body model diagram of the patient whose display orientation has been controlled. That is, the medical images of the patient are placed on the determined captured body parts of the human body model diagram at step S960, and the virtual human body model diagram of the patient on which the medical images of the patient have been placed is displayed at step S970.

In this case, the medical images of the patient to be placed on the human body model diagram may be adjusted to a predetermined scale, the scaled medical images may be placed on the corresponding captured body parts, and then the human body model diagram may be displayed. If any of the medical images does not accurately match a corresponding captured body part, a user may directly match them with each other.

The method in accordance with the embodiment of the present invention illustrated in FIG. 9 may be applied in conjunction with the technology of FIGS. 3 to 5. Through the above-described process, as illustrated in FIG. 10, information 1010 about a patient, a human body model diagram 1020 of the patient on which medical images have been placed, and thumbnail images 1030 may be displayed on a user viewer. It will be apparent that whether to display the thumbnail images may be determined by the user's selection.

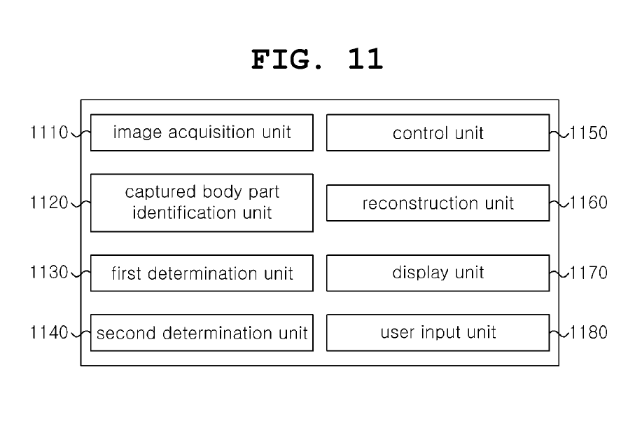

FIG. 11 illustrates the configuration of an apparatus for displaying medical images using a virtual patient model in accordance with an embodiment of the present invention.

Referring to FIG. 11, the apparatus in accordance with this embodiment of the present invention includes an image acquisition unit 1110, a captured body part determination unit 1120, a determination unit 1130, a checking unit 1140, control unit 1150, a reconstruction unit 1160, a display unit 1170, and a user input unit 1180.

The image acquisition unit 1110 acquires one or more medical images of a patient.

In this case, the image acquisition unit 1110 may acquire one or more medical images of the patient, for example, images of the patient taken by a CT scanner, an MRI scanner, a PET scanner, an angiographic machine, a CR/DR scanner, fundoscopy equipment, an EKG scanner or the like, via a PACS.

The captured body part determination unit 1120 determines the captured body part of each of the one or more medical images of the patient that are acquired by the image acquisition unit 1110.

In this case, the captured body part determination unit 1120 may determine the body part of the patient corresponding to each of the acquired medical images of the patient using medical image data stored for each of the medical images, for example, information stored in a DICOM header, or may determine the body part of the patient corresponding to each of the acquired medical images of the patient by performing image processing and analysis on each of the medical images of the patient.

The determination unit 1130 determines a human body model diagram corresponding to the unique information of the patient among a plurality of predetermined human body model diagrams.

In this case, the unique information of the patient may include information about the age, gender, height, and body weight of the patient, etc.

The checking unit 1140 determines whether a lesion is present (or exists) on the medical images of the patient.

The control unit 1150 controls the display orientation of the human body model diagram of the patient while taking into account the location of the lesion if, as a result of the determination by the checking unit 1140, it is determined that the lesion is present (or exists) on the medical images of the patient.

In this case, the control unit 1150 may determine one lesion among a plurality of lesions based on at least one of priorities predetermined based on the types of lesions and the field of profession of a user (doctor) if the plurality of lesions is present. It will be apparent that if a plurality of lesions is present, the notification of the presence of the lesions may be transferred to a user, and thus the user may directly select a lesion or a medical image including a lesion.

The reconstruction unit 1160 reconstructs the one or more medical images in accordance with the changed display orientation of the human body model diagram when the display orientation of the human body model diagram has been changed.

In this case, it is preferred that the reconstructed medical images be 3D medical images.

The display unit 1170 displays the human body model diagram of the patient with the medical images of the patient placed on the determined captured body parts.

When the display orientation of the human body model diagram of the patient has been changed, the display unit 1170 displays the human body model diagram whose display orientation has been changed or controlled with the reconstructed medical images placed on the respective captured body parts.

Furthermore, the display unit 1170 may display one of a plurality of medical images on a corresponding captured body part while taking into account at least one of the types and captured times of the images and predetermined priorities if the plurality of medical images corresponding to the same captured body part is present among the medical images of the patient, and may also display the number of the plurality of medical images on the corresponding region.

In this case, the display unit 1170 may display thumbnail images corresponding to a plurality of medical images in a separate display region if the plurality of medical images correspond to the same captured body part, or may display a predetermined diagram or color in a corresponding captured body part if the size of the captured body part of the medical image is smaller than a reference size.

The user input unit 1180 is configured to receive user input required for the present invention, such as a user input that enables a user to directly rotate a human body model diagram, a user input that enables a user to directly place a medical image, and a user input that enables a user to select a thumbnail image.

Additionally, the apparatus in accordance with the present invention may perform various functions that have been described in conjunction with the method of the present invention.

The method of displaying medical images using a virtual patient model in accordance with the embodiment of the present invention may be implemented in the form of program instructions that can be executed via various computer means and then stored in a computer-readable storage medium. The computer-readable storage medium may include program instructions, a data file, and a data structure solely or in combination. The program instructions that are stored in the medium may be designed and constructed particularly for the present invention, or may be known and available to those skilled in the field of computer software. Examples of the computer-readable storage medium include magnetic media such as a hard disk, a floppy disk and a magnetic tape, optical media such as CD-ROM and a DVD, magneto-optical media such as a floptical disk, and hardware devices particularly configured to store and execute program instructions such as ROM, RAM, and flash memory. Examples of the program instructions include not only machine language code that is constructed by a compiler but also high-level language code that can be executed by a computer using an interpreter or the like. The above-described hardware components may be configured to act as one or more software modules that perform the operation of the present invention, and vice versa.

The present invention is advantageous in that medical images of a patient are placed on a virtual human body model diagram suitable for the patient in accordance with captured body parts thereof, thereby enabling a user (a doctor) to intuitively check various and important images of the patient.

For example, captured medical images (CT, MR, PET, Angiography, CR/DR, fundoscopy, or EKG images) of an actual patient are placed on respective captured body parts of a virtual human body model diagram of the patient, and the display orientation of the human body model diagram is controlled by user input or depending on the location of a lesion included in the medical images of the patient, thereby enabling a user to easily check important images from a desired orientation. In this case, when the display orientation is changed, the 3D medical image are reconstructed and displayed in accordance with the display orientation, and thus the medical images of the patient can be easily checked and also a lesion can be easily checked.

Furthermore, the present invention is advantageous in that 3D medical images (MPR, MIP, or VR images, or the like) of a patient are placed and displayed on a human body model diagram of the patient, and thus the overall scanned status of the patient, the actual anatomical structure of the patient and the states of one or more lesions can be checked at a glance.

Furthermore, the present invention is advantageous in that if a plurality of medical images corresponds to the same captured body part, thumbnail images are separately provided, and thus the medical images of the same captured body part may be compared with each other or a desired image may be selected from among them.

Although the preferred embodiments of the present invention have been disclosed for illustrative purposes, those skilled in the art will appreciate that various modifications, additions and substitutions are possible without departing from the scope and spirit of the invention as disclosed in the accompanying claims.

What is claimed is:

1. A method of displaying medical images using a virtual patient model, comprising:
acquiring, by a processor, one or more medical images of a patient;
identifying, by the processor, a captured body part of each of the acquired one or more medical images;
determining, by the processor, whether a lesion exists on the one or more medical images;
controlling, by the processor, a display orientation of a predetermined human body model diagram of the patient while taking into account a location of the lesion if the lesion exists on the one or more medical images; and
placing and displaying, by the processor, the one or more medical images on the identified captured body parts of the human body model diagram, wherein display orientation of the human body model diagram has been controlled.

2. The method of claim 1, wherein the placing and displaying comprises:
reconstructing, by the processor, the one or more medical images in accordance with the display orientation of the human body model diagram; and
placing and displaying, by the processor, the reconstructed one or more medical images on the identified captured body parts.

3. The method of claim 1, wherein the placing and displaying comprises:
adjusting, by the processor, a size of each of the one or more medical images to a predetermined scale; and
placing and displaying, by the processor, the one or more medical images on the identified captured body parts.

4. The method of claim 1, wherein the placing and displaying comprises:
if a plurality of medical images being captured for a same body part, placing and displaying, by the processor, one of the plurality of medical images on the same body part while taking into account at least one of types of images, imaging times of the images and predetermined priorities.

5. The method of claim 4, wherein the placing and displaying one of the plurality of medical images comprises:
displaying, by the processor, a number of the plurality of medical images.

6. The method of claim 4, wherein the placing and displaying one of the plurality of medical images comprises:
displaying, by the processor, thumbnail images corresponding to the plurality of medical images in a separate display region.

7. The method of claim 1, wherein the placing and displaying comprises:
displaying, by the processor, a predetermined diagram or color in the captured body part if a size of the captured body part is smaller than a reference size.

8. The method of claim 1, wherein the controlling of the display orientation comprises:
if a plurality of lesions exists, determining, by the processor, one lesion among the plurality of lesions while taking into account at least one of predetermined priorities which is predetermined based on types of lesions and a field of profession of a user, and
controlling, by the processor, the display orientation of the human body model diagram while taking into account a location of the determined lesion.

9. The method of claim 1, further comprising determining, by the processor, a human body model diagram corresponding to unique information of the patient among a plurality of predetermined human body model diagrams;
wherein the controlling comprises controlling a display orientation of the determined human body model diagram corresponding to the unique information of the patient.

10. A method of displaying medical images using a virtual patient model, comprising:
acquiring, by a processor, one or more medical images of a patient;
identifying, by the processor, a captured body part of each of the acquired one or more medical images;
determining, by the processor, a human body model diagram corresponding to unique information of the patient among a plurality of predetermined human body model diagrams; and
placing and displaying, by the processor, the one or more medical images on the identified captured body parts of the determined human body model diagram.

11. The method of claim 10, wherein:
the determined human body model diagram can be rotated by a user input; and
the placing and displaying comprises:
if the display orientation of the human body model diagram is changed by the user, reconstructing the one or more medical images in accordance with the changed display orientation; and
disposing and displaying, by the processor, the reconstructed one or more medical images on the identified captured body parts.

12. The method of claim 10, further comprising:
determining, by the processor, whether a lesion exists on the one or more medical images; and
controlling, by the processor, a display orientation of the determined human body model diagram while taking into account a location of the lesion if a lesion exists on the one or more medical images;
wherein the placing and displaying places and displaces the one or more medical images on the identified captured body parts of the human body model diagram, wherein the display orientation of the human body model diagram has been controlled.

13. An apparatus for displaying medical images using a virtual patient model, comprising:
a processor configured to:
acquire one or more medical images of a patient;
identify a captured body part of each of the acquired one or more medical images;
determine a human body model diagram corresponding to unique information of the patient among a plurality of predetermined human body model diagrams; and
place and display the one or more medical images on the identified captured body parts of the determined human body model diagram.

14. The apparatus of claim 13, wherein the processor is further configured to:
determine whether a lesion exists on the one or more medical images;
control a display orientation of the determined human body model diagram while taking into account a location of the lesion if the lesion exist on the one or more medical images; and
place and display the one or more medical images on the identified captured body parts of the human body model diagram, wherein display orientation of the human body model has been controlled.

15. The apparatus of claim 14, wherein the processor is further configured to:
if the display orientation of the human body model diagram is changed by the user input, reconstruct the one or more medical images in accordance with the changed display orientation; and
place and display the reconstructed one or more medical images on the identified captured body parts.

16. The apparatus of claim 13, wherein the processor is further configured to:
display one of a plurality of medical images on the captured body part if the plurality of medical images captured for the same captured body part, while taking into account at least one of types of images, imaging times of the images and predetermined priorities, and
display a number of the plurality of medical images.

17. The apparatus of claim 13, wherein the processor is further configured to display thumbnail images corresponding to a plurality of medical images in a separate display region if the plurality of medical images captured for a same body part.

18. The apparatus of claim 13, wherein the processor is further configured to display a predetermined diagram or color in the captured body part if a size of the captured body part is smaller than a reference size.
